(19) United States
(12) Patent Application Publication
(10) Pub. No.: US 2003/0018044 A1
(43) Pub. Date: Jan. 23, 2003

(54) Treatment of ocular disease

(76) Inventor: Gholam A. Peyman, New Orleans, LA (US)

Correspondence Address: Beverly A. Lyman, Wood, Herron & Evans, L.L.P., 2700 Carew Tower, 441 Vine Street, Cincinnaty, OH, 45202-2917, US

(21) Appl. No.: 10/247,220

(22) Filed: Sep. 19, 2002

Related U.S. Application Data
(63) Continuation-in-part of application No. 09/507,076, filed on Feb. 18, 2000, now Pat. No. 6,489,335.

(51) Int. Cl. A61K 31/4745

(52) U.S. Cl. 514/291

(57) ABSTRACT

A formulation to treat ocular disease such as dry eye disease, as well as other diseases, is disclosed. Tacrolimus is administered intraocularly, such as topically or by injection. For topical administration, an amount of about 1 ng to 10 μg may be formulated in an aqueous based cream that may be applied at bedtime or throughout the day. For injection, a dose of about 20-1000 μg/ml is used. Tacrolimus may also be administered in milligram quantities as a surgical implant contained in a diffusible walled reservoir sutured to the wall of the sclera, or may be contained within an inert carrier such as microspheres or liposomes to provide a slow-release drug delivery system.

This application is a Continuation-In-Part application of U.S. patent application Ser. No. 09/507,076 filed Feb. 18, 2000, now pending.

### FIELD OF THE INVENTION

The invention is directed to therapeutic treatment of ocular diseases such as dry eye disease.

### BACKGROUND

Dry eye disease encompasses any condition where the tear film loses water and becomes more concentrated. It is a common complaint, affecting three million people in the United States alone, yet it is difficult to diagnose and treat. The loss of water from the tear film causes a corresponding rise in tear osmolarity. The increased osmolarity results in symptoms such as a sandy-gritty feeling in the eye, burning, irritation, or a foreign-body sensation that worsens during the day. Patients suffering from dry eye disease complain of mild to severe symptoms, with signs ranging from minimal superficial punctate keratitis to corneal perforation.

Dry eye disease has a chronic remitting and relapsing nature and may result from a number of factors. The disease may be a natural part of the aging process, affecting 15%-20% of adults over age 40. It may also result from pathological processes such as diseases of the lacrimal glands, mucus glands, and/or lipid producing glands, and may occur with cell infiltration or atrophy of the lacrimal gland (Sjogren's Syndrome). Estrogen deficiency in postmenopausal women is also postulated to result in dry eye disease.

One method to treat dry eye disease is by topical administration of over-the-counter drugs that serve as artificial tears. Numerous varieties of these artificial tears are available (TheraTears® (Advanced Vision Research), Refresh® and Celluvisc® (Allergan), Tears Natural® and Bion Tears® (Alcon), GenTeal® and HypoTears® (CIBA Vision), each of which contain electrolytes and has varying pH levels, osmolarities, and surface tensions. Another method to treat dry eye disease is by surgery to close the lacrimal drainage ducts using punctum plugs. Neither method, however, is completely desirable. Artificial tears do not have a constant flow rate as do human tears, and treat the symptoms rather the cause of the disease. Surgery has its attendant risks, and may not be a viable option in older patients.

It is known that Cyclosporin A (cyclosporine, Allergan Inc.), may treat dry eye disease since patients administered cyclosporine for other disorders have shown a marked increase in tear flow. A topical formulation containing Cyclosporin A (Arrestase®, Allergan Inc.) is currently under review by the Food and Drug Administration. Cyclosporin A is an immunomodulator, suggesting that immune-mediated inflammation contributes to dry eye disease. Cyclosporin A has been used to treat various ocular pathologies such as glaucoma, corticosteroid-induced ocular hypertension, allograft rejection, infections, and ocular surface disease. It is also known that Cyclosporin A may be used in the eye to treat uveitis (inflammation of the uvea) by topical, intravitreal or systemic administration. Doses of 0.05%, 0.1%, and 0.5% cyclosporine have been reported. Cyclosporin A has good penetration into the cornea but not into the anterior chamber, and does not increase intraocular pressure or cause cataracts.

Tacrolimus (Prograf®, previously known as FK-506) is an immunomodulating drug that has been applied topically to treat a variety of dermatoses. Topical administration of tacrolimus at doses ranging from 0.03%-0.3% resulted in significant clinical improvement in atopic dermatitis after 2-3 weeks treatment, and tacrolimus treatment of other dermatologic diseases shows promise. Tacrolimus, like cyclosporine, blocks the signal transduction pathway needed to induce interleukin-2 gene expression and thereby activate T lymphocytes. In addition to suppressing T cell activation, tacrolimus inhibits anti-IgE-triggered histamine release and inhibits prostaglandin D2 synthesis in human skin mast cells. While oral administration produces limiting adverse effects (systemic immunosuppression, infection, neural toxicity, nephrotoxicity, and hypertension), topical administration for treatment of dermatoses at concentrations up to 0.3% showed no significant difference in effects between treated and control groups. In addition, tacrolimus is well tolerated locally and only occasionally causes mild irritation.

The non-systemic use of tacrolimus in the treatment of ocular diseases including dry eye disease would be advantageous.

### SUMMARY OF THE INVENTION

The invention is directed to a method of treating ocular disease, such as dry eye disease, age related macular degeneration, retinitis pigmentosa, diabetic retinopathy, uveitis, scleritis, neuritis, and/or papilitis, by providing an effective amount of tacrolimus in a pharmaceutically acceptable formulation directly to a diseased eye. In one embodiment, the formulation is applied topically. In an alternative embodiment, the formulation is injected intraocularly, for example by subconjuctival, intravitreal, subretinal, or retrobulbar injection. For subconjunctival injection a concentration in the range of about 1 ng/ml to about 500 μg/ml tacrolimus may be used. For intravitreal injection a concentration in the range of about 1 μg/0.1 ml to about 1000 μg/0.1 ml may be used, with a preferred concentration of about 50 μg/0.1 ml tacrolimus. For subretinal injection, a concentration in the range of about 1 μg/0.1 ml to about 100 μg/0.1 ml may be used. For retrobulbar injection, a concentration in the range of about 20 μg/ml to about 1000 μg/ml tacrolimus may be used. Tacrolimus may be administered in an aqueous-based solution, for example tacrolimus bound to liposomes, or tacrolimus dissolved in an organic solvent. Tacrolimus may also be provided in an inert physiologically acceptable carrier by surgical implantation, injection, or topical application.

The invention is also directed to a composition for treating dry eye disease. The composition contains an effective amount of tacrolimus in a pharmaceutically acceptable formulation. The formulation may be an aqueous cream or liquid containing, for example, about 1 ng to 10 μg tacrolimus. The formulation may be an inert carrier such as a microsphere, liposome or polymeric matrix containing tacrolimus. Tacrolimus may be dissolved in an aqueous solvent such as 0.9% saline or 5% dextrose, or an organic solvent such as dimethylsulfoxide (DMSO) or an alcohol.

The invention is additionally directed to a composition for intraocular injection to treat ocular disease. An effective amount of tacrolimus is dissolved in either an aqueous solvent such as 0.9% saline or 5% dextrose, or an organic solvent such as DMSO or alcohol.

The invention is also directed to a method to treat a diseased eye by intraocular administration of a composition containing tacrolimus as the active agent. The composition is a pharmaceutically acceptable formulation, and contains an amount of tacrolimus that is effective to treat the diseased eye without substantial toxicity. The patient may be a diabetic and suffer from diabetic retinopathy, or may have an age related disease (e.g., dry eye disease, macular degeneration), or other disease such as retinitis pigmentosa. The composition may be administered topically (e.g., applied as a cream or gel, or as eye drops), or may be injected into the eye, or may be implanted in a device in or on the eye. Topical administration can be performed by a non-medical professional or by the patient, while injection and implantation are performed by medical professionals. An implant may be provide a time-release form of tacrolimus.

The invention is also directed to a method to reduce the onset or progression of diabetic retinopathy, age related macular degeneration, retinitis pigmentosa, or other ocular disease, by intraocularly administering a composition containing tacrolimus in a pharmaceutically acceptable formulation and in an effective amount without causing substantial toxicity. The composition may also contain Cyclosporin A, and may be applied topically, injected, or implanted.

The invention is also directed to a composition that contains tacrolimus as the active agent for treating a diseased eye without causing substantial toxicity to the eye. It may be a cream, a liquid that can be administered as drops or injected into the eye, or a capsule or other type of matrix that can be implanted on or in the eye.

These and other embodiments of the invention will be further appreciated with reference to the following detailed description.

### DETAILED DESCRIPTION

The invention is directed to a method and composition to treat ocular diseases by administration of tacrolimus (Prograf®, previously known as FK506). Tacrolimus, a macrolide immunosuppressant produced by Streptomyces tsukubaensis, is a tricyclo hydrophobic compound that is practically insoluble in water, but is freely soluble in ethanol and is very soluble in methanol and chloroform. It is available under prescription as either capsules for oral administration or as a sterile solution for intravenous administration. The solution contains the equivalent of 5 mg anhydrous tacrolimus in 1 ml of polyoxyl 60 hydrogenated castor oil (HCO-60), 200 mg, and dehydrated alcohol (USP, 80.0%^{v/v}), and must be diluted with a solution of 0.9% NaCl or 5% dextrose before use.

Tacrolimus may be administered in a topical formulation for treatment of ocular disease. In one embodiment, tacrolimus in amounts ranging from 1 ng to 10 μg is contained in an aqueous-based cream excipient. The drug may be incorporated directly into the cream in the same solution as used for intravenous administration, or may be contained in liposomes or microspheres either in solution or in an anhydrous form. The cream formulation is usually applied to the eye at bedtime, but it may be applied any time throughout the day if the cream does not cause blurred vision. Tacrolimus may also be applied topically in the form of eye drops using the same solution for intravenous administration.

Tacrolimus may also be injected intraocularly, using intravitreal (into the vitreous), subconjunctival (into the subconjunctiva), subraretinal (under the retina), or retrobulbar (behind the eyeball) injection. For subconjuctival injection, a dose in the range of about 1 ng/ml to about 500 μg/ml may be used. For intravitreal injection, a dose in the range of about 1 μg/0.1 ml to about 1000 μg/0.1 ml my be used, with a preferred dose of 50 μg/0.1 ml. For retrobulbar injection, a dose in the range of about 20 μg/ml to about 1000 μg/ml may be used. For subretinal injection, a dose in the range of about 1 μg/0.1 ml to about 100 μg/0.1 ml may be used.

The toxicity of tacrolimus, which had been reported with tacrolimus administered systemically, was thought to limit its intraocular use. The toxicity of tacrolimus administered intraocularly had not been evaluated. Ocular toxicity may manifest as a gross and/or histologic retinal and/orvitreous toxic reaction. Evidence of such a toxic reaction may include one or more of white vitreous bodies, white vitreous opacities, electroretinography abnormalities such as reduction in mean B-wave amplitude in both scotopic and photopic conditions, occlusion of the temporal retinal vessels, and fibrin deposits.

In one embodiment, tacrolimus is administered in an amount or at a dose that does not result in substantial toxicity to the eye. As used herein, a lack of substantial toxicity encompasses both the absence of any manifestations of toxicity, as well as manifestations of toxicity which one skilled in the art would consider not sufficiently detrimental to decrease or cease treatment. As one example, fibrin deposits may be present indicating some toxicity, but less than substantial toxicity if their duration, number, etc., as determined by one skilled in the art, does not warrant that treatment be stopped. As another example, white vitreous bodies and fibrin bodies may be present indicating some toxicity, but less than substantial toxicity if their duration, number, etc., as determined by one skilled in the art, does not warrant that treatment be stopped.

It was surprisingly found that direct intraocular injection of a dose up to about 250 μg tacrolimus occurs without substantial toxicity to the patient. The intravenous solution form of tacrolimus may be diluted to achieve the indicated concentration using 0.9% NaCl or 5% dextrose, or an organic solvent such as dimethylsulfoxide (DMSO) or alcohol, preferably a low molecular weight alcohol. Intraocular administration may be any of the routes and formulations previously described. For injection, either a solution, emulsion, suspension of a liquid, capsular formulation of microspheres or liposomes, etc. may be used.

Tacrolimus may also be administered surgically as an ocular implant. As one example, a reservoir container having a diffusible wall of polyvinyl alcohol or polyvinyl acetate and containing milligram quantities of tacrolimus may be implanted in the sclera. As another example, tacrolimus in milligram quantities may be incorporated into a polymeric matrix having dimensions of about 2 mm by 4 mm, and made of a polymer such as polycaprolactone, poly(glycolic) acid, poly(lactic) acid, or a polyanhydride, or a lipid such as sebacic acid, and may be implanted on the sclera or in the eye. This is usually accomplished with the patient receiving either a topical or local anesthetic and using a small (3-4 mm incision) made behind the cornea. The matrix, containing tacrolimus, is then inserted through the incision and sutured to the sclera using 9-0 nylon.

Tacrolimus may also be contained within an inert matrix for either topical application or injection into the eye. As one example of an inert matrix, liposomes may be prepared from dipalmitoyl phosphatidylcholine (DPPC), preferably prepared from egg phosphatidylcholine (PC) since this lipid has a low heat transition. Liposomes are made using standard procedures as known to one skilled in the art. Tacrolimus, in amounts ranging from nanogram to microgram quantities, is added to a solution of egg PC, and the lipophilic drug binds to the liposome.

A time-release drug delivery system may be implanted intraocularly to result in sustained release of the active agent over a period of time. The implantable formation may be in the form of a capsule of any of the polymers previously disclosed (e.g., polycaprolactone, poly(glycolic) acid, poly(lactic) acid, polyanhydride) or lipids that may be formulation as microspheres. As an illustrative example, tacrolimus may be mixed with polyvinyl alcohol (PVA), the mixture then dried and coated with ethylene vinyl acetate, then cooled again with PVA. Tacrolimus bound with liposomes may be applied topically, either in the form of drops or as an aqueous based cream, or may be injected intraocularly. In a formulation for topical application, the drug is slowly released overtime as the liposome capsule degrades due to wear and tear from the eye surface. In a formulation for intraocular injection, the liposome capsule degrades due to cellular digestion. Both of these formulations provide advantages of a slow release drug delivery system, allowing the patient a constant exposure to the drug over time.

In a time-release formulation, the microsphere, capsule, liposome, etc. may contain a concentration of tacrolimus that could be toxic if administered as a bolus dose. The time-release administration, however, is formulated so that the concentration released at any period of time does not exceed a toxic amount. This is accomplished, for example, through various formulations of the vehicle (coated or uncoated microsphere, coated or uncoated capsule, lipid or polymer components, unilamellar or multilamellar structure, and combinations of the above, etc.). Other variables may include the patient's pharmacokinetic-pharmacodynamic parameters (e.g., body mass, gender, plasma clearance rate, hepatic function, etc.). The formation and loading of microspheres, microcapsules, liposomes, etc. and their ocular implantation are standard techniques known by one skilled in the art, for example, the use a ganciclovir sustained-release implant to treat cytomegalovirus retinitis, disclosed in Vitreoretinal Surgical Techniques, Peyman et al., Eds. (Martin Dunitz. London 2001, chapter 45); Handbook of Pharmaceutical Controlled Release Technology, Wise, Ed. (Marcel Dekker, New York 2000), the relevant sections of which are incorporated by reference herein in their entirety.

As another example, tacrolimus may be dissolved in an organic solvent such as DMSO or alcohol as previously described and containing a polyanhydride, poly(glycolic) acid, poly(lactic) acid, or polycaprolactone polymer.

Tacrolimus, either alone or in combination with immunosuppressant agents such as Cyclosporin A, may be administered intraocularly and without substantial toxicity, to treat ocular disease such as retinopathy in diabetic patients, macular degeneration, retinitis pigmentosa, inflammatory diseases of the eye such as Behcet's syndrome, toxoplasmosis, Birdshot choroidopathy, histoplasmosis, pars planitis, sarcoidosis, inflammatory diseases of the choroid of unknown etiology such as sympathetic ophthalmia, serpiginous choroidopathy, diffuse pigment epitheliopathy, Vogt-Koyanagi syndrome, polyarteritis nodosa, juvenile rheumatic arthritis, other conditions of the eye including uveitis (inflammation of the uvea), scleritis, (inflammation of the sclera), neuritis (inflammation of the optic nerve), or papilitis (inflammation of the optic nerve head) using the methods and formulations previously described. This may be achieved by one or a combination of factors, such as by slowing disease progression, lessening its severity, lengthening the time of onset, etc.

To treat uveitis, tacrolimus is preferably injected subconjuctivally at a dose in the range of about 1 ng/ml to about 50 μg/ml, or intravitreally at a dose of about 1 μg/0.1 ml to about 1000 μg/0.1 ml, preferably about 50 μg/0.1 ml. To treat scleritis involving the anterior sclera, tacrolimus is preferably administered topically. To treat scleritis involving the posterior sclera, tacrolimus is preferably ad ministered by retrobulbar injection at a dose in the range of about 20 μg/ml to about 1000 μg/ml and dissolved in DMSO or a low concentration of alcohol. To treat neuritis or papilitis, tacrolimus is preferably administered by retrobulbar injection at a dose in the range of about 20 μg/ml to about 1000 μg/ml.

Diabetic retinopathy is a leading cause of blindness. Patients with diabetes mellitus have an absolute or relative lack of circulating insulin and, through a variety of factors, frequently present with vascular changes in the retina. These changes manifest in retinal microaneurysms, small hemorrhages, and exudates, and lead to the formation of scar tissue. New blood vessels may form around the optic disk (proliferative retinopathy). Over time, the cumulative results of such vascular effects lead to ocular pathologies which, ultimately, decrease vision in the diabetic patient. Thus, compositions and methods which reduce these vascular changes, or reduce their effects, improve the chances of a diabetic patient either maintaining vision, or at least slowing loss of vision.

Macular degeneration, also called age related macular degeneration (AMD) is a pathological condition that results in proliferation of new blood vessels in the subretinal area. While the presence of the new vessels themselves is not problematic, new vessels leak blood and other serous fluid which accumulate in surrounding spaces. It is this fluid accumulation that leads to visual impairment. For example, in the retina, both the large vessels and the capillaries normally have intact vessel walls. In the choroid, the large vessels normally have intact vessel walls, but the capillary walls or membranes contain fenestrations or openings. Any endogenous or exogenous fluid present in these capillaries, for example, blood, serous fluid, solubilized drug, etc. will leak outside the vessels and into the surrounding area. The accumulation of fluid can result in serous and hemorrhagic detachment of the retinal pigment epithelium and neurosensory retina, and can lead to loss of vision due to fibrous deform scarring. Patients with an early stage of AMD can be diagnosed by the presence in the eye of abnormal clumps of pigments, termed drusen, which are dead outer segments of photoreceptor cells under the retinal pigment epithelium. The presence of large, soft drusen in the eye indicates a pre-stage of exudative AMD, and places these patients at higher-than-average risk for developing neovascularizations, especially if one eye is already affected.

Retinitis pigmentosa is a general term that encompasses a disparate group of disorders of rods and cones, which are the sensory structures in the retina. While retinitis pigmentosa is a genetic disorder, and is not an inflammatory process, one manifestation of the disease is the presence of irregular black deposits of clumped pigment in the peripheral retina. Thus, there is likely at least some immune component to retinitis pigmentosa which treatment with an immunosuppressant drug may help to alleviate.

A possible mechanism for tacrolimus' therapeutic efficacy in ocular disease involves its immunosuppressant activity. For example, diabetic patients treated with immunosuppressant drugs for reasons unrelated to vision develop less retinopathy over time than other diabetic patients. As another example, the drusen that is present in AMD constitutes a chronic inflammatory stimulus that becomes the target for encapsulation by a variety of inflammatory mediators, such as compliment. Treatment with immunosuppressant drug may ameliorate this reaction. Immunosuppressant therapy results in decreased numbers of circulating immunocompetent cells such as lymphocytes. These cells otherwise have the potential to participate in an immune response, to lodge within the small capillaries and arterioles of the eye to form blockages and hence occlude blood flow, etc In addition to lymphocytes, other hematopoietic cells may also be affected by immunotherapy, and include erythrocytes (red blood cells), megakaryocytes (precursors to platelets) and thrombocytes (platelets), and other leukocytes (white blood cells), such as monocytes and granulocytes. Local or in situ administration of immunosuppressant agents to the eye would be expected to decrease the number of these cells, resulting in reduction in the immune response, less blockage, increased blood flow, and increased patency of the ocular vessels.

Tacrolimus in any of the previously described formulations, dosages, compositions, routes of administration, etc. may be employed. The active ingredient may be tacrolimus alone; alternatively, tacrolimus may be administered as described in combination with one or more known immunosuppressant agents, such as Cyclosporin A. Because the immunosuppressant(s) is administered locally to the eye (e.g., intraocular injection, topical ocular application), the undesirable effects brought about by administration of systemic immunosuppressant therapy (e.g., decreased peripheral blood leukocyte count, susceptibility to infections, hepatic and renal toxicity of the immunosuppressant agent itself, etc.) are absent.

It should be understood that the embodiments of the present invention shown and described in the specification are only preferred embodiments of the inventor who is skilled in the art and are not limiting in any way. Therefore, various changes, modifications or alterations to these embodiments may be made or resorted to without departing from the spirit of the invention and the scope of the following claims.

What is claimed is:

1. A method to treat a diseased eye in a patient comprising intraocularly administering to the patient a composition consisting essentially of tacrolimus in a pharmaceutically acceptable formulation and in an amount effective to treat the diseased eye without substantial toxicity to the patient.

2. The method of claim 1 to treat a disease selected from the group consisting of an age related disease, an inflammatory disease, a degenerative disease, and combinations thereof.

3. The method of claim 1 to treat a disease selected from the group consisting of dry eye disease, scleritis, neuritis, papillitis, uveitis, retinopathy, retinitis pigmentosa, macular degeneration, Behcet's syndrome, toxoplasmosis, Birdshot choroidopathy, histoplasmosis, pars planitis, sarcoidosis, sympathetic ophthalmia, serpiginous choroidopathy, diffuse pigment epitheliopathy, Vogt-Koyanagi syndrome, polyarteritis nodosa, and juvenile rheumatic arthritis.

4. The method of claim 1 wherein the composition is administered topically to the eye.

5. The method of claim 4 wherein the composition is administered as drop, a cream, or a gel.

6. The method of claim 1 wherein the composition is injected intraocularly.

7. The method of claim 1 wherein the composition is administered by retrobulbar, intravitreal, intraretinal, or subconjunctival injection.

8. The method of claim 1 wherein the composition is implanted intraocularly.

9. The method of claim 1 wherein the composition is implanted on the sclera.

10. The method of claim 1 wherein the composition is in a sustained release matrix implanted intraocularly.

11. The method of claim 10 wherein the matrix is sutured to the sclera.

12. The method of claim 1 wherein the composition contains Cyclosporin A.

13. The method of claim 1 wherein the composition is administered at a dose up to 250 μg tacrolimus.

14. The method of claim 7 wherein injection is subconjuctival at a dose in the range of about 1 ng/ml to about 500 μg/ml, intravitreal at a dose in the range of about 1 μg/0.1 ml to about 1000 μg/0.1 ml, retrobulbar at a dose in the range of about 20 μg/ml to about 1000 μg/ml, or subretinal at a dose in the range of about 1 μg/0.1 ml to about 100 μg/0.1 ml.

15. The method of claim 7 wherein injection is intravitreal at a dose of about 50 μg/0.1 ml.

16. A method to treat a diseased eye in a patient comprising intraocularly administering to the patient a composition consisting essentially of tacrolimus in a pharmaceutically acceptable formulation and in an amount effective to treat the diseased eye.

17. The method of claim 16 to treat a disease selected from the group consisting of an age related disease, an inflammatory disease, a degenerative disease, and combinations thereof.

18. The method of claim 16 to treat a disease selected from the group consisting of dry eye disease, scleritis, neuritis, papillitis, uveitis, retinopathy, retinitis pigmentosa, macular degeneration, Behcet's syndrome, toxoplasmosis, Birdshot choroidopathy, histoplasmosis, pars planitis, sarcoidosis, sympathetic ophthalmia, serpiginous choroidopathy, diffuse pigment epitheliopathy, Vogt-Koyanagi syndrome, polyarteritis nodosa, and juvenile rheumatic arthritis.

19. The method of claim 16 wherein the composition is administered topically to the eye.

20. The method of claim 16 wherein the composition is injected intraocularly.

21. The method of claim 20 wherein the composition is administered by subconjuctival injection at a dose in the range of about 1 ng/ml to about 500 μg/ml, intravitreal injection at a dose in the range of about 1 μg/0.1 ml to about 1000 μg/0.1 ml, retrobulbar injection at a dose in the range of about 20 μg/ml to about 1000 μg/ml, or subretinal injection at a dose in the range of about 1 μg/0.1 ml to about 100 μg/0.1 ml.

22. The method of claim 16 wherein the composition is implanted intraocularly.

23. The method of claim 22 wherein the composition is in a sustained release matrix implanted intraocularly.

24. The method of claim 16 wherein the composition contains Cyclosporin A.

25. A therapeutic composition for treating a diseased eye consisting essentially of tacrolimus in a physiologically acceptable intraocular formulation and in an amount effective to treat the diseased eye without substantial toxicity to the eye.

26. The composition of claim 25 formulated as a cream with tacrolimus in an amount ranging from about 1 ng to about 10 μg.

27. The composition of claim 25 formulated as an injectable with tacrolimus in the range of about 1 ng/ml to about 500 μg/ml for subconjuctival injection, about 1 μg/0.1 ml to about 1000 μg/0.1 ml for intravitreal injection, about 20 μg/ml to about 1000 μg/ml for retrobular injection, and about 1 μg/0.1 ml to about 100 μg/0.1 ml for subretinal injection.

28. The composition of claim 25 wherein tacrolimus is in or on a physiologically compatible inert matrix.

29. The composition of claim 28 wherein the matrix comprises a substance selected from the group consisting of lipid, polyvinyl alcohol, polyvinyl acetate, polycaprolactone, poly(glycolic)acid, poly(lactic)acid, and combinations thereof.

30. The composition of claim 28 wherein the matrix sustainedly releases tacrolimus.
